(19) United States
(12) Patent Application Publication
(10) Pub. No.: US 2025/0082773 A1
(43) Pub. Date: Mar. 13, 2025

(54) COMBINATION THERAPY USING EXOSOME SECRETION INHIBITOR AND IMMUNE CHECKPOINT INHIBITOR

(71) Applicant: Research & Business Foundation SUNGKYUNKWAN UNIVERSITY, Suwon-si, Gyeonggi-do (KR)

(72) Inventors: Jae Hyung PARK, Suwon-si, Gyeonggi-do (KR); Jung Min SHIN, Hwaseong-si, Gyeonggi-do (KR); Soyoung SON, Suwon-si, Gyeonggi-do (KR); Sol SHIN, Suwon-si, Gyeonggi-do (KR); Seok Ho SONG, Suwon-si, Gyeonggi-do (KR); Chanho KIM, Suwon-si, Gyeonggi-do (KR); Torsha GHOSH, Suwon-si, Gyeonggi-do (KR); Kyung Hee HAN, Seoul (KR); Jeongyun KIM, Suwon-si, Gyeonggi-do (KR)

(21) Appl. No.: 18/292,514

(22) PCT Filed: Jul. 28, 2022
(86) PCT No.: PCT/KR2022/011119

§ 371 Date: Jan. 26, 2024

(30) Foreign Application Priority Data
Jul. 28, 2021 (KR) 10-2021-0099362

(51) Int. Cl. A61K 47/68; A61K 31/42; A61K 31/506; A61K 39/00; A61K 47/60; A61P 35/00; C07K 16/28

(52) U.S. Cl. CPC A61K 47/6809; A61K 31/42; A61K 31/506; A61K 47/60; A61P 35/00; C07K 16/2818; C07K 16/2827; A61K 2039/505

(57) ABSTRACT

A drug combination, an antibody-drug conjugate, and a polymer-drug conjugate are disclosed, for use in the combination therapy that uses as an exosome secretion inhibitor an endothelin receptor type A (ETA) antagonist, and an immune checkpoint inhibitor.

### TECHNICAL FIELD

Disclosed are drug combinations, antibody-drug conjugates, and polymer-drug conjugates for use in combination therapy using ETA (Endothelin receptor type A) antagonists as exosome secretion inhibitors and immune checkpoint inhibitors.

### BACKGROUND ART

Immunotherapy, one of the most potent strategies for the treatment of cancer, provides excellent clinical benefits by modulating the immune system of the body to boost innate antitumor activity. Thus, cancer immunotherapies (e.g., cancer vaccination, immune checkpoint blockade, or chimeric antigen receptor (CAR)-T cell therapy) have recently emerged as promising alternatives to conventional treatments (e.g., chemotherapy, surgery, and radiation) and candidates for combinatorial approaches. In particular, immune checkpoint inhibitors are considered as one of the most promising therapeutic options because they have been shown to cause significant tumor remission in the clinic in various cancer types such as melanoma, breast cancer, and lung cancer. Unlike autologous dendritic cell-based vaccines and CAR-T cells, immune checkpoint inhibitors can be mass-produced and are available to all cancer patients.

In immune checkpoint therapy, cancer patients are treated with monoclonal antibodies against specific immune checkpoint molecules, such as PD-L1, PD-1, and CTLA-4. Once the negative regulation by the immune checkpoint is inhibited, the function of cytotoxic T cells is reinvigorated, which then eliminates cancer cells leading to the remission of tumors. However, a considerable proportion of cancer patients (>70%) do not respond to immune checkpoint inhibitors because cancer cells often create immunosuppressive microenvironments as part of their immune escape mechanisms.

### DISCLOSURE

### Technical Problem

To address this limitation of immune checkpoint therapy, it is necessary to develop a novel therapeutic approach that can boost cytotoxic T cells and neutralize the immune escape mechanisms of cancer.

### Technical Solution

Exosomes (50-200 nm in diameter) produced by most eukaryotic cells play a critical role in intercellular communication by interacting with the receptors or delivering bioactive cargos into the recipient cells. To exhaust of the CD8⁺ cytotoxic T cells, tumor cells not only express PD-L1 on their surface, but also secrete exosomal PD-L1 through the fusion of multivesicular bodies with the plasma membrane. Although elevated levels of IFN-7 increase PD-L1 expression in cancer cells, immune checkpoint inhibitors, such as anti-PD-1 antibodies, effectively bind to PD-1 in circulating CD8⁺ cytotoxic T cells, leading to its effective antitumor efficacy. In contrast, exosomal PD-L1 binds to circulating CD8⁺ cytotoxic T cells in the blood, exhausting the CD8⁺ cytotoxic T cells. Thus, in the presence of exosomal PD-L1, Immune checkpoint inhibitors, such as anti-PD-1 antibodies, are no longer bound to CD8⁺ cytotoxic T cells, resulting in diminished therapeutic efficacy. In the present invention, it was found that an ETA antagonist can significantly increase the response rate to immune checkpoint therapy by suppressing the secretion of cancer exosomes and switching non-responders to immune checkpoint inhibitors into responders. Specifically, in the present invention, it was found that ETA antagonist significantly decreased exosomal PD-L1 levels in blood and activated CD8⁺ cytotoxic T cells when combined with immune checkpoint inhibitor in animal models. These findings imply that ETA antagonist modulates the immunosuppressive tumor microenvironment (TME) by inhibiting of exosomal PD-L1 and can thus be used as a potential agent to increase the reactivity of immune checkpoint inhibitor. In addition, the present invention demonstrated that an ETA antagonist can be used in combination therapy as an antibody-drug conjugate or polymer-drug conjugate.

Therefore, an embodiment described herein provides a combination for the prevention or treatment of cancer comprising an endothelin receptor type A (ETA) antagonist and an immune checkpoint inhibitor, wherein the ETA antagonist and the immune checkpoint inhibitor are administered simultaneously, separately, or sequentially.

The present invention also provides a method for treating cancer comprising administering an ETA antagonist and an immune checkpoint inhibitor to a subject in need thereof, wherein the ETA antagonist and the immune checkpoint inhibitor are administered simultaneously, separately, or sequentially.

The present invention also provides a use of an ETA antagonist and an immune checkpoint inhibitor in the manufacture of medicine for the treatment of cancer, wherein the ETA antagonist and the immune checkpoint inhibitor may be administered simultaneously, separately, or sequentially.

Another embodiment described herein provides a composition for preventing or treating cancer comprising a conjugate of an ETA antagonist conjugated to a biocompatible polymer, wherein the composition is administered simultaneously, separately, or sequentially with the administration of the immune checkpoint inhibitor to a patient receiving the immune checkpoint inhibitor.

The present invention also provides a method for treating cancer comprising administering a conjugate of an ETA antagonist conjugated to a biocompatible polymer to a subject in need thereof, wherein the conjugate is administered simultaneously, separately, or sequentially with the administration of the immune checkpoint inhibitor to a patient receiving the immune checkpoint inhibitor.

The present invention also provides a use of a conjugate of an ETA antagonist conjugated to a biocompatible polymer in the manufacture of medicine for the treatment of cancer, wherein the conjugate may be administered simultaneously, separately, or sequentially with the administration of the immune checkpoint inhibitor to a patient receiving the immune checkpoint inhibitor.

Another embodiment described herein provides a composition for preventing or treating cancer comprising a conjugate of an ETA antagonist conjugated to an immune checkpoint inhibitor.

The present invention also provides a method for treating cancer comprising administering a conjugate of an ETA antagonist conjugated to an immune checkpoint inhibitor to a subject in need thereof.

The present invention also provides a use of a conjugate of an ETA antagonist conjugated to an immune checkpoint inhibitor in the manufacture of medicine for the treatment of cancer.

### BRIEF DESCRIPTION OF DRAWINGS

FIG. 1: Schematic illustration depicting the mechanism of action of combination therapy using SFX and αPD-1. SFX, an FDA-approved ETA antagonist, inhibits cancer exosome biogenesis and synergistically enhances the antitumor effect of αPD-1. (1) Tumors actively secrete exosome with PD-L1 (exosomal PD-L1), which inhibits T cell activation as an immune escape mechanism in αPD-1 monotherapy. (2) SFX inhibits exosome biogenesis in tumors, leading to enhanced antitumor efficacy of αPD-1.

FIG. 2: Quantification of exosomal PD-L1 in plasma from CT26 tumor-bearing mice. (A) Experimental regime of exosomal PD-L1 isolation. (B) Relative exosomal PD-L1 in plasma from WT and CT26 tumor-bearing mice (n=7 and 11 for WT and CT26, respectively).

FIG. 3: SFX synergistically enhances the antitumor effect of an immune checkpoint inhibitor. (A) Schematic illustration of the therapeutic schedule for CT26 tumor-bearing mice. (B) Antitumor effects of SFX, αPD-1, and SFX+αPD-1. Average tumor volume (left) and individual tumor volumes of mice (right) (n=10).

FIG. 4: (A) Photographs of the tumors harvested on day 21 (n=10). (B) Tumor weight after treatment. (C) Quantification of exosomal PD-L1 in mouse plasma after therapeutic regime. (D) Cytokine levels in plasma were quantified using ELISA (n=6). *p<0.05, **p<0.01, ***p<0.001. Error bar, SD.

FIG. 5: Antitumor efficacy of combinational treatment of SFX and αPD-L1 in CT26 tumor-bearing mice. (A) Experimental scheme for antitumor efficacy. (B) Average tumor volume (n=7). (C) Individual tumor volume (n=7).

FIG. 6: Combination of SFX and αPD-1 elicits adaptive immunity against tumor. (A) Schematic illustration of the therapeutic schedule for CT26 tumor-bearing mice. (B) Representative histogram of CD45⁺CD4⁺ cells in tumor microenvironment (TME). (C) Quantification of CD45⁺CD4⁺ cells in the TME (n=3). (D) Representative histogram of CD45⁺CD8⁺ cells in TME. (E) Quantification of CD45⁺CD4⁺ cells in TME (n=5) (F) Representative dot plot of CD45⁺CD3⁺CD8⁺ cytotoxic T cells in the TME. (G) Quantification of CD45⁺CD3⁺CD8⁺ cytotoxic T cells in the TME (n=9). *p<0.05, **p<0.01, ***p<0.001. Error bar, SD.

FIG. 7: Ability of ETA antagonists to inhibit exosome secretion. (A) Ability of ETA antagonists to inhibit exosome secretion in CT26 cell line. (B) Ability of ETA antagonists to inhibit exosome secretion in B16F10 cell line.

FIG. 8: Results of evaluating the therapeutic efficacy of Ab-VC-AMB conjugate according to one embodiment described herein in a disease animal model. (A) Schematic illustration of the therapeutic schedule for CT26 tumor-bearing mice. (B) Antitumor efficacy of AMB, αPD-L1 and ADC. Average tumor volume (left) and individual tumor volumes of mice (right) (n=4). (C) Tumor weight after treatment. (D) Photographs of the tumors harvested on day 22 (n=4).

FIG. 9: Results of evaluating the ability of Ab-VC-AMB conjugate according to one embodiment described herein to inhibit exosome secretion in disease animal model. (A) Schematic illustration of an experiment to isolate exosomes from plasma of an animal model. (B) Results of quantifying PD-L1 on the surface of isolated exosomes.

FIG. 10: Synthesis strategy of PEG-b-Poly(L-lysine-CDM-SFX) according to one embodiment described herein.

FIG. 11: Confirmation of preparation of PEG-b-Poly(L-lysine) through ¹H NMR.

FIG. 12: Confirmation of introduction of pH-sensitive linker through ¹H NMR.

FIG. 13: Confirmation of preparation of PEG-b-Poly(L-lysine-CDM-SFX) through ¹H NMR.

FIG. 14: Results of evaluating the ability of PEG-b-Poly (L-lysine-CDM-SFX) according to one embodiment described herein to inhibit exosome secretion through nanoparticle tracking analysis (NTA)

FIG. 15: Ability of ETA antagonists to inhibit exosome secretion in CT26 cell line.

FIG. 16: BST synergistically enhances the antitumor effect of an immune checkpoint inhibitor. (A) Schematic illustration of the therapeutic schedule for CT26 tumor-bearing mice. (B) Average tumor volume (n=9). (C) Individual tumor volumes of mice (n=9). (D) Tumor weight after treatment. *p<0.05, **p<0.01, ***p<0.001. Error bar, SD.

FIG. 17: MCT synergistically enhances the antitumor effect of an immune checkpoint inhibitor. (A) Schematic illustration of the therapeutic schedule for CT26 tumor-bearing mice. (B) Average tumor volume (n=6). (C) Individual tumor volumes of mice (n=6).

FIG. 18: Synthesis strategy of PEG-SS-AMB according to one embodiment described herein FIG. 19: Results of evaluating the ability of PEG-SS-AMB to inhibit exosome secretion in the CT26 murine colon cancer cell line through nanoparticle tracking analysis (NTA) FIG. 20: Results of evaluating the therapeutic efficacy of PEG-SS-AMB conjugate according to one embodiment described herein in a disease animal model. (A) Schematic illustration of the therapeutic schedule for CT26 tumor-bearing mice. (B) Average tumor volume (n=5). (C) Individual tumor volumes of mice (n=5) (D) Photographs of the tumors harvested on day 21 (n=5).

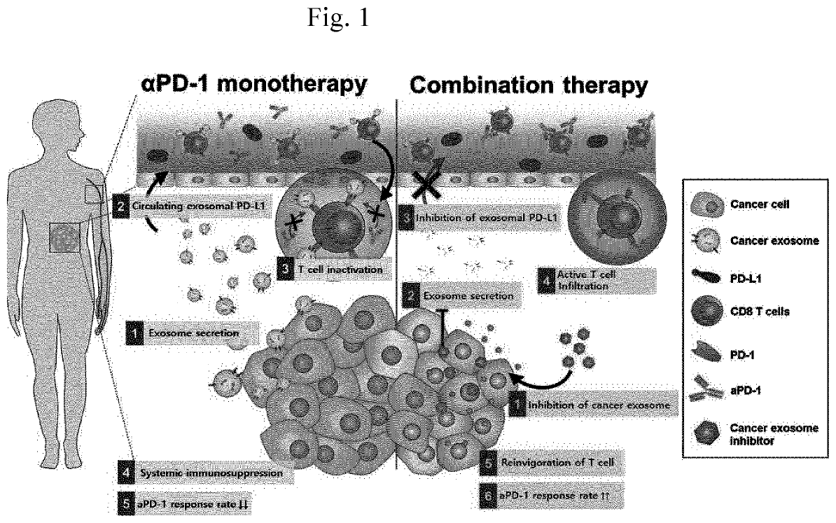

### BEST MODE FOR CARRYING OUT THE INVENTION

In one embodiment, the present invention relates to a use of a combination of an endothelin receptor type A (ETA) antagonist and an immune checkpoint inhibitor to prevent or treat cancer. In particular, the present invention provides a combination for the prevention or treatment of cancer comprising an endothelin receptor type A (ETA) antagonist and an immune checkpoint inhibitor, wherein the ETA antagonist and the immune checkpoint inhibitor are administered simultaneously, separately, or sequentially. Further, the present invention provides a method for treating cancer comprising administering an ETA antagonist and an immune checkpoint inhibitor to a subject in need thereof, or a use of an ETA antagonist and an immune checkpoint inhibitor in the manufacture of medicine for the treatment of cancer, wherein the ETA antagonist and the immune checkpoint inhibitor may be administered simultaneously, separately, or sequentially.

In a preferred embodiment, the ETA antagonist may be selected from the group consisting of ambrisentan, sulfisoxazole, macitentan, BQ-123, BQ-788, zibotentan, sitaxentan, atrasentan, bosentan, tezosentan and A192621.

In a preferred embodiment, the immune checkpoint inhibitor may be an antibody that specifically binds to PD-1 or PD-L1, or antigen-binding fragment thereof.

In a preferred embodiment, the ETA antagonist may be a conjugate conjugated to a biocompatible polymer.

Herein, the biocompatible polymer may be a polymer comprising a nonionic hydrophilic polymer moiety, a polymer comprising an ionic polymer moiety, or a copolymer comprising a nonionic hydrophilic polymer moiety and an ionic polymer moiety.

The nonionic hydrophilic polymer may be polyethylene glycol, polypropylene glycol, polyoxazoline, polyvinylpyrrolidone, polyvinyl alcohol, polyacrylamide, polymethacrylamide, polyacrylic acid ester, polymethacrylic acid ester, polyhydroxyethyl methacrylate, dextran, polysaccharide, or methylcellulose.

The ionic polymer may be poly(L-lysine), polyaspartic acid, poly(L-glutamic acid), polyornithine, polyarginine, polyhomoarginine, polyhistidine, hyaluronic acid, alginic acid, polyacrylic acid, polymethacrylic acid, chitosan, polyethyleneimine, polyvinyl phosphate, polyethylene glycol methacrylate phosphate, carboxymethylcellulose, or heparin.

In one embodiment, the ETA antagonist may be conjugated to a biocompatible polymer via a linker or is conjugated to a biocompatible polymer via a pH-sensitive linker or an acid-labile linker.

Alternatively, the linker may be a cleavable linker that is cleaved by a protease.

The copolymer may be a block copolymer or a graft copolymer.

In a preferred embodiment, the ETA antagonist may be in the form of a conjugate conjugated to an immune checkpoint inhibitor.

In one embodiment, the ETA antagonist is conjugated to the immune checkpoint inhibitor via a linker or is conjugated to a biocompatible polymer via a pH-sensitive linker or an acid-labile linker.

In another embodiment, the present invention relates to a use of a conjugate of an ETA antagonist conjugated to a biocompatible polymer for the prevention or treatment of cancer. In particular, the present invention provides a composition for preventing or treating cancer comprising a conjugate of an ETA antagonist conjugated to a biocompatible polymer, wherein the composition is administered simultaneously, separately, or sequentially with the administration of the immune checkpoint inhibitor to a patient receiving the immune checkpoint inhibitor. Further, the present invention provides a method for treating cancer comprising administering a conjugate of an ETA antagonist conjugated to a biocompatible polymer to a subject in need thereof, or a use of a conjugate of an ETA antagonist conjugated to a biocompatible polymer in the manufacture of medicine for the treatment of cancer, wherein the conjugate may be administered simultaneously, separately, or sequentially with the administration of the immune checkpoint inhibitor to a patient receiving the immune checkpoint inhibitor.

In another embodiment, the present invention relates to a use of a conjugate of an ETA antagonist conjugated to an immune checkpoint inhibitor. In particular, the present invention provides a composition for preventing or treating cancer comprising a conjugate of an ETA antagonist conjugated to an immune checkpoint inhibitor. Further, the present invention provides a method for treating cancer comprising administering a conjugate of an ETA antagonist conjugated to an immune checkpoint inhibitor to a subject in need thereof, or a use of a conjugate of an ETA antagonist conjugated to an immune checkpoint inhibitor in the manufacture of medicine for the treatment of cancer.

Hereinafter, the present invention will be described in more detail.

Where the terms “comprise”, “comprises”, “comprised” or “comprising” are used in this specification (including the claims) they are to be interpreted as specifying the presence of the stated features, integers, steps or components, but not precluding the presence of one or more other feature, integer, step, component or group thereof.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention before the priority date of each claim of this application.

The term “immune checkpoint” refers to a mechanism that turns the immune response on or off to control uncontrolled immune responses under normal physiological conditions. An immune checkpoint is classified into a stimulatory immune checkpoint that increases the immune response and an inhibitory immune checkpoint that suppresses the immune response. An inhibitory immune checkpoint stimulates immune checkpoint proteins to control excessive immune responses and reduce immune cell activity, but cancer cells use this mechanism to avoid attacks by immune cells. For example, specific proteins expressed on the surface of cancer cells bind to proteins on the surface of immune cells, thereby inhibiting immune cells from attacking cancer cells. For example, programmed death-ligand 1 (PD-L1) expressed on the surface of cancer cells binds to programmed cell death protein 1 (PD-1) present on the surface of T cells and inhibits T cell function. In addition to PD-1 and PD-L1, non-limiting examples of inhibitory immune checkpoint proteins include cytotoxic T-lymphocyte-associated protein 4 (CTLA-4) and its ligand B7.1/2 (1CD80/CD86); indoleamine-pyrrole 2,3-dioxygenase (IDO1); T cell membrane protein (TIM, e.g. TIM3); adenosine A2a receptor (A2aR); lymphocyte activation gene (LAG, e.g. LAG3); killer immunoglobulin receptor (KIR), etc.

The term “immune checkpoint inhibitor” refers to substances that inhibit immune checkpoints and has a mechanism to reinvigorates T cells by binding to the binding site between cancer cells and T cells and blocking immune evasion signals. For example, antibodies that block the binding of PD-1 to PD-L1 by binding to either PD-1 or PD-L1 allow T-cells to attack tumors.

In one embodiment, the immune checkpoint inhibitor may be an antibody that specifically binds to PD-1 or PD-L1, or antigen-binding fragment thereof. Examples of antibodies that specifically bind to PD-1 include Pembrolizumab, Nivolumab, or Cemiplimab, and antibodies that specifically bind to PD-L1 include Atezolizumab, Avelumab, or Durvalumab, but are not limited thereto, and any antibody or antigen-binding fragment thereof that specifically binds to PD-1 or PD-L1 is included in the scope of the present application.

The term “antibody” collectively refers to a protein that specifically binds to a specific antigen, and is used in the broadest sense, and it may be a protein produced in the immune system by stimulation of an antigen or a protein synthesized chemically or prepared recombinantly. The type thereof is not particularly limited. Specifically, the antibody encompasses monoclonal antibodies (including full-length monoclonal antibodies), polyclonal antibodies, multispecific antibodies (e.g., bispecific antibodies), synthetic antibodies (also called antibody mimetics), chimeric antibodies, humanized antibodies, human antibodies, or antibody fusion proteins (also called antibody conjugates), provided such antibodies exhibit a desired biological activity.

An intact antibody (e.g., IgG-type) has a structure having two full-length light chains and two full-length heavy chains, each light chain associated with the heavy chain through disulfide bonds. A constant region of an antibody is divided into a heavy chain constant region and a light chain constant region, wherein the heavy chain constant region has a gamma (γ), mu (μ), alpha (α), delta (δ), or epsilon (ε) type, and a subclass of gamma1 (γ1), gamma2 (γ2), gamma3 (γ3), gamma4 (γ4), alpha1 (α1), or alpha2 (α2), and the light chain constant region has a kappa (κ) or lambda (λ) type.

The term “antigen-binding fragment” refers to a fragment of an antibody, which is able to specifically bind to an antigen even though at least part of amino acids present in the full-length chain is absent. Such a fragment is biologically active in that it binds to a target antigen, and competes with other antigen-binding molecules including an intact antibody for binding to a given epitope. The antigen-binding fragment may not include a constant heavy chain domain (i.e., CH2, CH3, and CH4 according to antibody isotypes) of Fc region of an intact antibody. Examples of the antigen-binding fragment may include single chain variable fragments (scFvs) (e.g., scFv, (scFv)₂, etc.), fragment antigen binding (Fab) (e.g., Fab, Fab′, F(ab′)₂, etc.), domain antibodies, peptibodies, minibodies, intrabodies, diabodies, triabodies, tetrabodies, or and single-chain antibodies, etc., but are not limited thereto. Further, the antigen-binding fragment may be scFv, or a fusion polypeptide (scFv-Fc), in which scFv is fused with an Fc region of an immunoglobulin (e.g., IgA, IgD, IgE, IgG (IgG1, IgG2, IgG3, IgG4), IgM, etc.), or a fusion polypeptide (scFv-Ck (kappa constant region) or scFv-Cλ (lambda constant region), in which scFv is fused with a light chain constant region (e.g., kappa or lambda), but is not limited thereto.

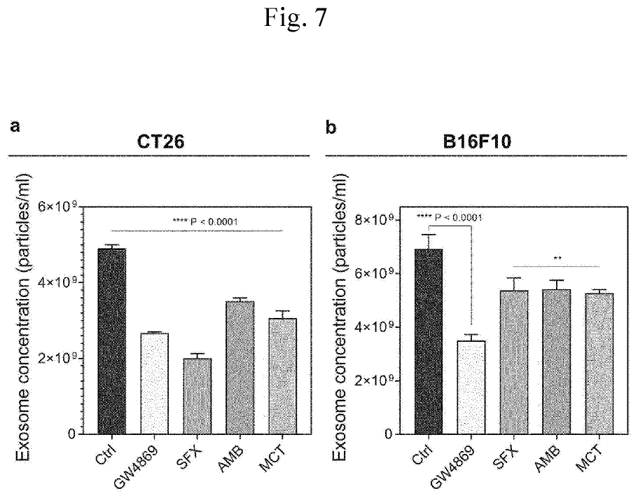

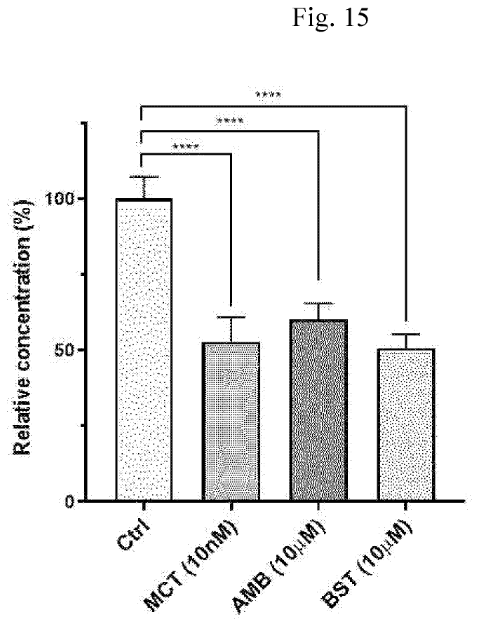

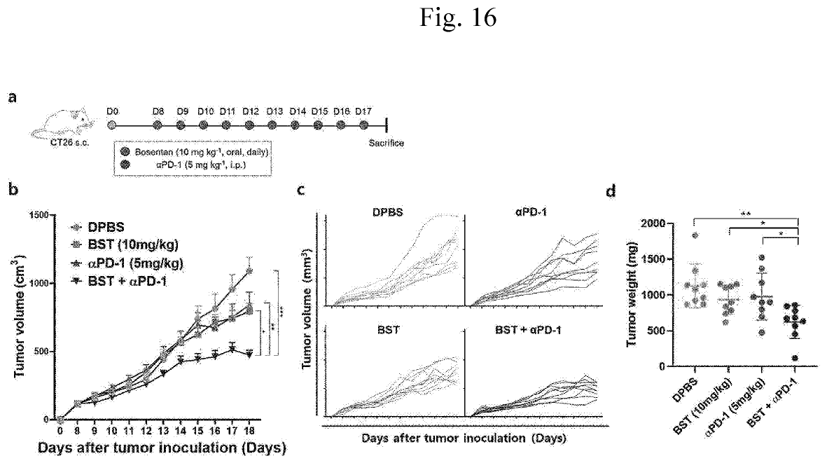

The term “Endothelin receptor type A (ETA) antagonist” refers to a substance that acts on the endothelin receptor molecule and inhibits or suppresses its function. For example, sulfisoxazole (SFX), an FDA-approved ETA antagonist, is known to inhibit tumor growth and metastasis by targeting endothelin receptor A (ETA) and inhibiting cancer exosome secretion (E. J. Im et al., Nat. Commun. 10, 1387 (2019)). In the present invention, it was confirmed that not only sulfisoxazole (SFX) inhibits cancer exosome secretion, but also other ETA antagonists effectively inhibit exosome secretion (FIG. 7 and FIG. 15).

In the present invention, it was found that an ETA antagonist can significantly increase the response rate to immune checkpoint therapy by suppressing the secretion of cancer exosomes and switching non-responders to immune checkpoint inhibitors into responders. Specifically, in the present invention, it was found that ETA antagonist significantly decreased exosomal PD-L1 levels in blood and activated CD8⁺ cytotoxic T cells when combined with immune checkpoint inhibitor. These findings imply that ETA antagonist modulates the immunosuppressive tumor microenvironment (TME) by inhibiting of exosomal PD-L1 and can thus be used as a potential agent to increase the reactivity of immune checkpoint inhibitor.

In one embodiment, the ETA antagonist may be selected from the group consisting of ambrisentan, sulfisoxazole, macitentan, BQ-123, BQ-788, zibotentan, sitaxentan, atrasentan, bosentan, tezosentan and A192621, but are not limited thereto.

Herein, combined administration of an ETA antagonist and an immune checkpoint inhibitor shows a synergistic cancer treatment effect.

The cancer may be a solid cancer or a blood cancer. Non-limiting examples thereof may include breast cancer, lung cancer, prostate cancer, ovarian cancer, brain cancer, liver cancer, cervical cancer, endometrial cancer, uterine cancer, colon cancer, colorectal cancer, colorectal cancer, rectal cancer, kidney cancer, nephroblastoma, skin cancer, oral squamous cell carcinoma, epidermoid carcinoma, nasopharyngeal cancer, head and neck cancer, bone cancer, esophageal cancer, bladder cancer, lymphoma (e.g., Hodgkin's lymphoma or non-Hodgkin's lymphoma), gastric cancer, pancreatic cancer, testicular cancer, thyroid cancer, follicular carcinoma, melanoma, myeloma, multiple myeloma, mesothelioma, osteosarcoma, myelodysplastic syndrome, tumor of mesenchymal origin, soft tissue sarcoma, liposarcoma, gastrointestinal stromal sarcoma, malignant peripheral nerve sheath tumor (MPNST), Ewing's sarcoma, leiomyosarcoma, mesenchymal chondrosarcoma, lymphosarcoma, fibrosarcoma, rhabdomyosarcoma, teratocarcinoma, neuroblastoma, medulloblastoma, glioma, benign skin tumor, or leukemia. The lung cancer may be, for example, small cell lung carcinoma (SCLC) or non-small cell lung carcinoma (NSCLC). The leukemia may be, for example, acute myeloid leukemia (AML), chronic myelogenous leukemia (CML), acute lymphocytic leukemia (ALL), or chronic lymphocytic leukemia (CLL).

In one embodiment, the ETA antagonist and immune checkpoint inhibitor may be administered simultaneously, separately, or sequentially.

In another embodiment, the ETA antagonist is administered in the form of a polymer-drug conjugate (PDC) conjugated to a biocompatible polymer, and the immune checkpoint inhibitor may be administered simultaneously, separately, or sequentially.

In another embodiment, the ETA antagonist may be administered in the form of an antibody-drug conjugate (ADC) conjugated to an immune checkpoint inhibitor.

The term “biocompatibility” refers to a property that is not substantially toxic to the human body, is chemically inactive, and can be compatible with good affinity with living tissues or living systems without causing inflammatory reactions, immune reactions or carcinogenesis. Covalent bonding of a polymer to a drug can change the surface properties and solubility of the molecule, providing many advantages such as increasing solubility in water or organic solvents, reducing immunoreactivity, or increasing stability in vivo, or prolonging elimination by the intestinal system, kidneys, spleen, or liver.

In the present invention, ETA antagonist can be conjugated to a biocompatible polymer. Biocompatible polymer can increase the half-life of a drug, improve cancer targeting, or improve the physical properties, stability, or bioavailability of a drug.

Non-limiting examples of biocompatible polymers herein include polyethylene glycol, polypropylene glycol, polyoxyethylene, polytrimethylene glycol, polylactic acid and its derivatives, polyacrylic acid and its derivatives, polyamino acid, polyvinyl alcohol, polyurethane, polyphosphazine, poly(L-lysine), polyalkylene oxide, polysaccharide, dextran, polyvinylpyrrolidone, or polyacrylamide, or non-immunogenic polymer material composed of two or more copolymers selected from the above polymers. Biocompatible polymers include not only linear but also branched polymers.

Other examples of biocompatible polymers herein include polymers a polymer comprising a nonionic hydrophilic polymer moiety, a polymer comprising an ionic polymer moiety, or a copolymer comprising both.

The nonionic hydrophilic polymer may be polyethylene glycol, polypropylene glycol, polyoxazoline, polyvinylpyrrolidone, polyvinyl alcohol, polyacrylamide, polymethacrylamide, polyacrylic acid ester, polymethacrylic acid ester, polyhydroxyethyl methacrylate, dextran, polysaccharide, or methylcellulose, but is not limited thereto.

The ionic polymer may be poly(L-lysine), polyaspartic acid, poly(L-glutamic acid), polyornithine, polyarginine, polyhomoarginine, polyhistidine, hyaluronic acid, alginic acid, polyacrylic acid, polymethacrylic acid, chitosan, polyethyleneimine, polyvinyl phosphate, polyethylene glycol methacrylate phosphate, carboxymethylcellulose, or heparin, but is not limited thereto.

The copolymer may be a block copolymer or a graft copolymer, but is not limited thereto. When using a PEG-derived block (polyoxyethylene chain block), the molecular weight of the PEG block may be about 1.0 to 100 kDa, 2 to 80 kDa, or 8 to 25 kDa, but is not limited thereto. Further, the number of repeating units of oxyethylene in the PEG block may be 2 to 3000, 20 to 2000, or 100 to 1000, but is not limited thereto. In a preferred embodiment, the copolymer may be a polyethylene glycol-block-poly(L-lysine) copolymer, but is not limited thereto.

The ETA antagonist and the biocompatible polymer may be linked via a linker. Further, the ETA antagonist and the immune checkpoint inhibitor may be linked via a linker. The linker may be designed as a cleavable linker which is cleaved in a cancer microenvironment. By cleavage of the linker, the ETA antagonist may be released from the biocompatible polymer, or the ETA antagonist and the immune checkpoint inhibitor may be released respectively. The cleavable linker may be a linker designed to be cleaved in response to characteristic elements of the cancer microenvironment (pH, ROS, enzymes, hypoxia, etc.), which are distinguished from normal tissues. Therefore, in a preferred embodiment of the present application, the ADC or PDC is linked via a tumor-specific linker, and after administration, the linker is cleaved to effectively deliver the ETA antagonist and immune checkpoint inhibitor to cancer cells, thereby maximizing the inhibition of secretion of tumor-derived exosomes and improving the efficacy of combined treatment with immune checkpoint inhibitors.

In one embodiment, the cleavable linker may be pH responsive, that is, responsive to hydrolysis at a specific pH value. Typically, the pH-responsive linker is hydrolysable under an acidic condition. For example, an acid-labile linker, which is hydrolysable in lysosomes (e.g., hydrazone, semicarbazone, thiosemicarbazone, cis-aconitic amide, orthoester, acetal, or ketal) may be used. As another example, a dimethyl maleic anhydride derivative, such as 2-propionic-3-methylmaleic anhydride (carboxylated dimethyl maleic anhydride or CDM) may be used. Such a linker is relatively stable under neutral pH conditions, for example, under pH conditions in blood, it may be unstable at an acidic pH of the tumor microenvironment, so it can be cleaved.

In another embodiment, the linker may be a cleavable linker which is cleaved by a protease. For example, the protease may be an intracellular peptidase or protease, as well as a lysosome or endosome protease, and may be, for example, cathepsin B, cathepsin K, matrix metalloproteinase (MMP), urokinase, or plasmin, but the present invention is not limited thereto. The linker may be a peptide linker. Peptides that are constituents of the peptide linker may include 20 major amino acids and minor amino acids well known in the field of biochemistry, for example, two or more amino acid residues, including citrulline. The amino acid residue includes all stereoisomers and may be in a D or L steric configuration. For example, the peptide may be an amino acid unit including 2 to 12 amino acid residues independently selected from glycine, alanine, phenylalanine, lysine, arginine, valine and citrulline. As an exemplary peptide linker, a Val-Cit linker or a Phe-Lys dipeptide may be included.

The linker may include a spacer moiety for binding the linker to an antibody. For example, the linker may include a reactive moiety having an electrophilic group that is reactive to a nucleophilic group on an antibody as a spacer moiety. The electrophilic group on the linker provides a convenient linker attachment site for the antibody. A useful nucleophilic group on an antibody includes, for example, sulfhydryl, a hydroxyl group and an amino group. A heteroatom of the nucleophilic group of the antibody is reactive to the electrophilic group on the linker and forms a covalent bond to the linker. A useful electrophilic group of the linker includes, for example, maleimide (for example, maleimidocaproyl) and a haloacetamide group.

Further, the linker may include a reactive moiety having a nucleophilic group that is reactive to an electrophilic group on an antibody as a spacer moiety. The nucleophilic group on the linker provide a convenient attachment site for the linker. A useful electrophilic group on an antibody includes, for example, aldehyde, a ketone carbonyl group and a carboxylic acid group. A heteroatom of the nucleophilic group of the linker may react with the electrophilic group on the antibody and may form a covalent bond to the antibody. A nucleophilic group of the linker includes, for example, hydrazide, oxime, amino, hydrazine, thiosemicarbazone, hydrazine carboxylate and arylhydrazide. The nucleophilic group on the linker provides a convenient attachment site for the linker.

Additionally, the linker of the present invention may include a self-immolative moiety (for example, p-aminobenzyl alcohol (PABA), p-aminobenzyloxycarbonyl (PABC), PAB-OH, and the like).

Administration of the combination or composition herein can prevent a disease, or inhibit, stop, or delay the onset or progression of a disease state, or improve or beneficially modify symptoms.

The term “effective amount” refers to an amount sufficient to achieve the desired result, e.g., an amount effective to treat or prevent cancer, when administered to subjects, including humans. The effective amount may vary depending on various factors such as a formulation method, administration mode, a patient's age, body weight, sex, disease severity, diet, administration time, administration route, excretion rate, and response sensitivity. Administration dosage or therapeutic regimen may be adjusted to provide an optimal therapeutic response as will be understood by those skilled in the art.

The combination or composition of the present disclosure may be provided together with one or more additives selected from the group consisting of pharmaceutically acceptable carriers, diluents, and excipients.

The pharmaceutically acceptable carrier, which is commonly used in the formulation of antibody, may be, for example, one or more selected from the group consisting of lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methylhydroxybenzoate, propylhydroxybenzoate, talc, magnesium stearate, mineral oil, etc., but is not limited thereto. The combination or composition may further include one or more selected from the group consisting of diluents, excipients, lubricants, wetting agents, sweeteners, flavoring agents, emulsifiers, suspending agents, preservatives, etc., which are commonly used in the preparation of pharmaceutical compositions, in addition to the above components. Pharmaceutically acceptable carriers and formulations suitable for the present invention, including those exemplified above, are described in detail in Remington's Pharmaceutical Sciences, current edition.

The combination or composition may be administered orally or parenterally. When administered parenterally, intravenous injection, subcutaneous injection, intramuscular injection, intraperitoneal injection, endothelial administration, topical administration, intranasal administration, intraocular administration, intrathecal administration, intrathecal administration, intracranial administration, intrastriatal administration may be used.

In some embodiments, the composition is provided as a sterile liquid preparation, for example, as an isotonic aqueous solution, a suspension, an emulsion, a dispersion, or a viscous composition, which, in some aspects, may be buffered to a selected pH. Liquid preparations are normally easier to prepare than gels, other viscous compositions, and solid compositions. In addition, liquid compositions are particularly convenient to administer by injection. Viscous compositions, on the other hand, may be formulated within the appropriate viscosity range to provide longer contact periods with a specific tissue. Liquid or viscous compositions may include a carrier which may be a solvent or dispersion medium containing, for example, water, saline, phosphate buffered saline, polyol (e.g., glycerol, propylene glycol, liquid polyethylene glycol) and appropriate mixtures thereof.

Sterile injectable solutions may be prepared by incorporating the binding molecule in a solvent, such as in admixture with a suitable carrier, diluent, or excipient such as sterile water, physiological saline, glucose, dextrose, or the like. The compositions may also be lyophilized. The compositions may include auxiliary substances such as wetting, dispersing, or emulsifying agents (e.g., methylcellulose), pH buffering agents, gelling or viscosity enhancing additives, preservatives, flavoring agents, colors, etc., depending upon the route of administration and the desired preparation.

Various additives which enhance stability and sterility of the compositions, including antimicrobial preservatives, antioxidants, chelating agents, and buffers, may be added.

Prevention of microbial actions may be ensured by various antibacterial and antifungal agents, for example, parabens, chlorobutanol, phenol, sorbic acid, etc. Prolonged absorption of the injectable pharmaceutical form may be brought about by the use of agents delaying absorption, for example, aluminum monostearate and gelatin.

### Detailed Description of the Embodiments

Hereinafter, the present invention will be described in more detail with reference to the following examples. However, these examples are only for illustrating the present invention, and the scope of the present invention is not limited by these examples.

### Example 1. Evaluation of Synergistic Effects of Combined Use of ETA Antagonist and Immune Checkpoint Inhibitor

### 1-1. Materials and Methods

### (1) Materials

anti PD-1 antibody (hereinafter, αPD-1) was purchased from BioXCell (Lebanon, NH, USA), sulfisoxazole (hereinafter, SFX) was purchased from Sigma Aldrich (St. Louis, MO, USA), bosentan (hereinafter, BST), macitentan (hereinafter, MCT) and ambrisentan (hereinafter, AMB) were purchased from Ambeed, respectively. anti PD-L1 antibody (hereinafter, αPD-L1) was purchased from eBioscience (14-5983-82, San Diego, CA, USA). The deionized water used in this study was purified using the AquaMax-Ultra Water Purification System (Anyang, Republic of Korea). All other chemicals were used as received without further purification.

### (2) Cell Lines and Cell Culture

Murine melanoma B16F10 cells and murine colon cancer CT26 cells were obtained from the American Type Culture Collection (Manassas, VA, USA). B16F10 cells were cultured in Dulbecco's modified Eagle's medium (DMEM) supplemented with 10% fetal bovine serum (FBS) and 1% antibiotic/antimycotic solution. CT26 cells were cultured in RPMI supplemented with 10% FBS and 1% antibiotic/antimycotic solution.

### (3) Establishment of Animal Model

All animal procedures were approved by the Institutional Animal Care and Use Committees of the Sungkyunkwan University (SKKUIACUC2020-05-15-2) and Kyungpook National University (KNUIACUC2020-0016). CT26 cells (2×10⁶) were suspended in cold PBS and subcutaneously injected to establish CT26 tumor-bearing mice.

### (4) Isolation and Quantitation of EXO

Exosomes (hereinafter, EXOs) were purified by differential centrifugation. Briefly, cell supernatants were subjected to differential centrifugation at 300×g/3 min, 2,500×g/15 min, and 10,000×g/30 min. After filtration through a 0.22 m filter, the supernatant was centrifuged at 120,000×g for 90 min. The pellets were re-suspended with phosphate-buffered saline (PBS) and centrifuged at 120,000×g/90 min again. The pellet (containing EXOs) was resuspended in PBS or RIPA lysis buffer for further analysis.

Mouse plasma EXOs were centrifuged at 2,500×g for 15 min and 10,000×g for 30 min to remove cells and cell debris. The supernatant was then centrifuged at 120,000×g for 90 min

The EXO proteins were quantified using the Pierce BCA Protein Assay kit (Thermo Scientific, Waltham, MA, USA) after treatment with RIPA buffer [Cell Signaling Technology (CST), Danvers, MA, USA].

### (5) Nanoparticle Tracking Analysis (NTA)

The number of EXOs was measured using NTA as described in our previous study (J. E. Lee, et al., J. Proteomics 131, 17-28 (2016)). Suspensions containing EXOs from cell culture medium were analyzed using a NanoSight LM10 instrument (NanoSight, Wiltshire, UK). For this analysis, a monochromatic laser beam (405 nm) was applied to a dilute suspension of the EXOs. A video of 30-s duration was recorded at a rate of 30 frames/s, and EXO movement was analyzed using NTA software (version 2.2; NanoSight). NTA post-acquisition settings were optimized and kept constant between samples, and each video was analyzed to estimate the concentration.

### (6) Tumor Growth Inhibition Test

After establishing CT26 tumor-bearing mice, the mice were treated with Dulbecco's phosphate buffered saline (DPBS), ETA antagonist (SFX, BST or MCT), immune checkpoint inhibitor (αPD-1 or αPD-L1), or ETA antagonist+immune checkpoint inhibitor (immune checkpoint inhibitor: oral administration, ETA antagonist: intraperitoneal administration) when the average tumor volume reached 50 mm³ or 100 mm³. The tumor volume was measured using calipers and calculated for each mouse using the following equation: V=½ab² (a is the longest axis and b is the shortest axis). After the treatment schedule, the tumors were excised and weighed.

### (7) Detection of PD-L1 on EXOs

To isolate circulating mouse EXOs, blood samples from the CT26 tumor-bearing mice were collected at the end of the antitumor efficacy study. Plasma was isolated via centrifugation at 2,000×g for 20 min, and the cell-free plasma was centrifuged at 16,500×g for 45 min to remove microvesicles. EXOs were isolated using a total EXO isolation kit (Invitrogen, Cat #4484450, Carlsbad, CA, USA). To assess PD-L1 on EXOs isolated from mouse plasma, ELISA plates were coated with a monoclonal antibody against PD-L1 (R&D Systems, Minneapolis, MN, USA) overnight at 25° C. Free binding sites were blocked with blocking buffer for 2 h at 25° C. After washing the plates with 0.05% Tween-20 in PBS, EXOs were added to each well and incubated for 2 h at 25° C. The EXO-containing wells were then sequentially incubated with the biotinylated PD-L1 antibody for 2 h and horseradish peroxidase-conjugated streptavidin for 20 min at 25° C. The plate was incubated for 20 min with a substrate solution composed of H₂O₂ and tetramethylbenzidine. After the addition of a stop solution containing 2N H₂SO₄ (R&D Systems, DY994), the plate was immediately read at 450 nm using an xMark™ microplate reader (Bio-Rad).

### (8) Flow Cytometric Analysis

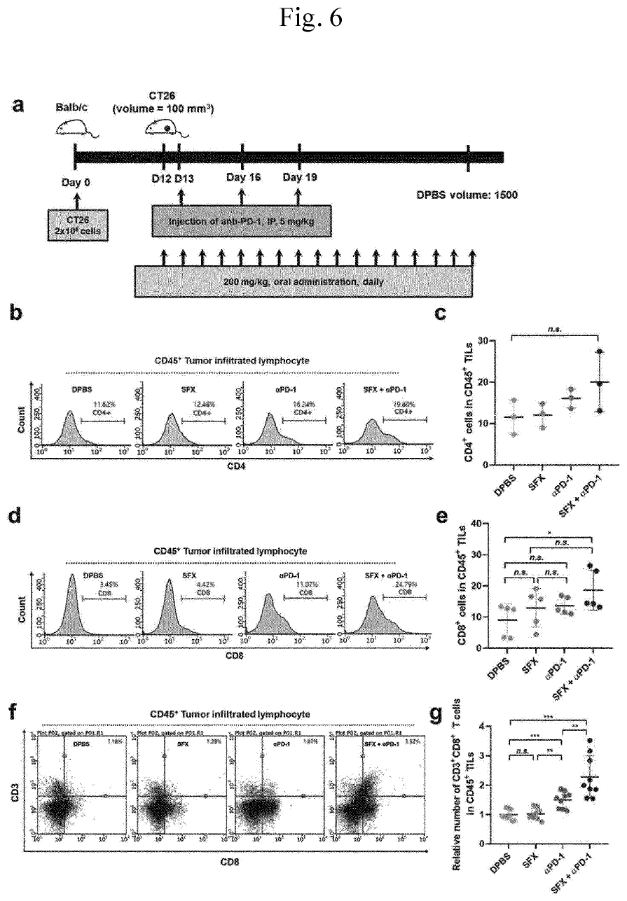

Following the therapeutic schedule (as described in FIG. 6A), the tumor tissue was removed and a single-cell suspension was obtained using the gentleMACS™ Tumor Dissociation Kit (Miltenyi Biotec), according to the manufacturer's instructions. Then, CD45⁺ TILs were isolated using MACS beads (MicroBeads, Miltenyi Biotec). The isolated TILs were labeled with CD3 (PE labeled, Biolegend, San Diego, CA, USA), CD8 (FITC-labeled, Biolegend), or CD4 (FITC-labeled, Biolegend) antibodies. The cells were then analyzed using Guava easyCyte (EMD Millipore, Billerica, MA, USA).

### (9) Statistical Analysis

Statistical significance of the experimental results was assessed using one-way analysis of variance (ANOVA) or unpaired two-tailed Student's t-test. Error bars in the graphical data represent mean±standard deviation. All in vitro experiments were performed in triplicate unless otherwise stated. A p-value <0.05 was regarded as statistically significant (indicated with an asterisk (*) in the corresponding figures as follows: *p<0.05, **p<0.01, ***p<0.001).

### 1-2. Results

### (1) SFX Reinvigorates T Cell Activity by Inhibitinm Cancer EXO Secretion

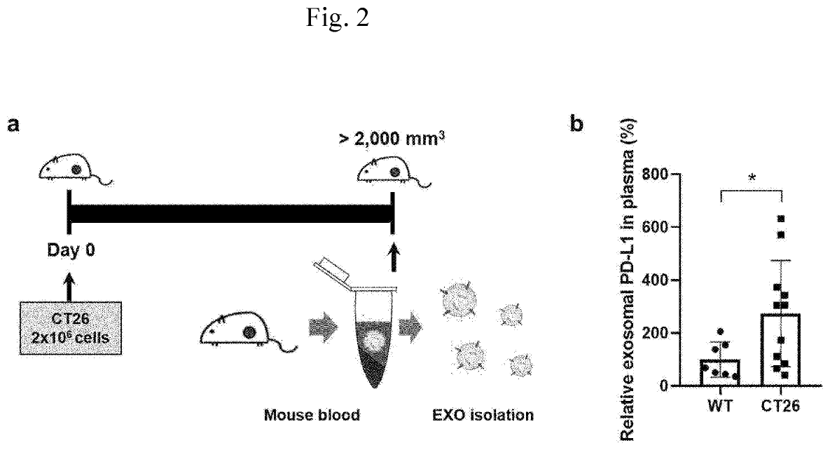

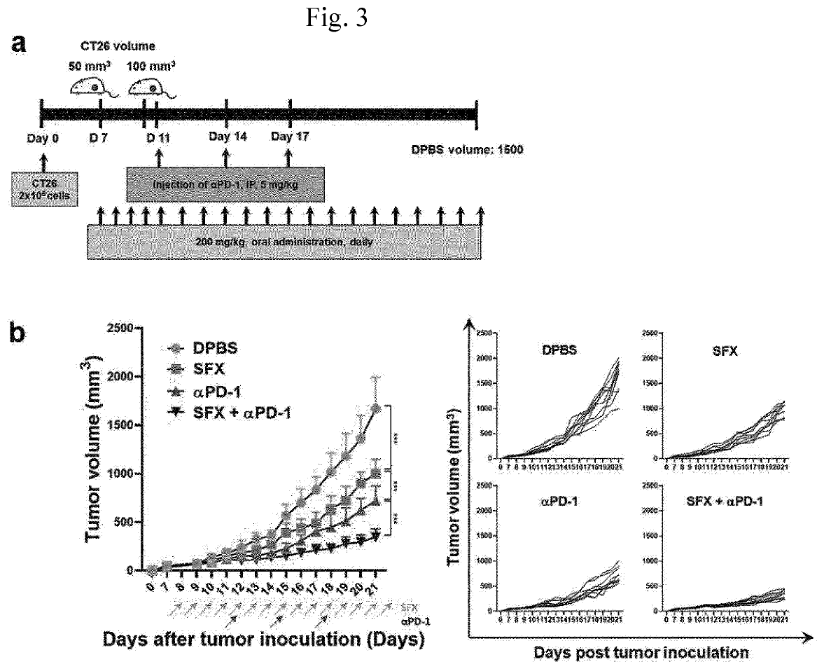

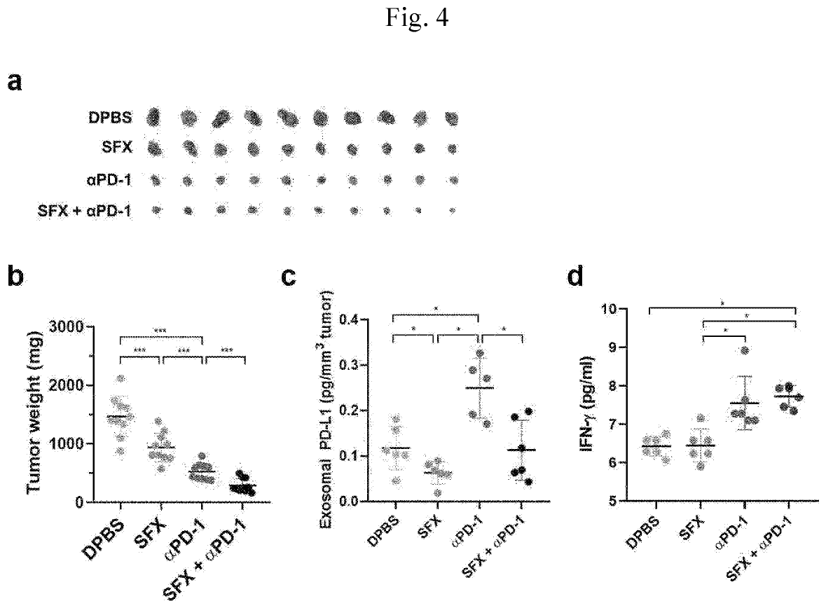

EXO-mediated immunosuppression is primarily based on the interaction between PD-L1 on tumor-derived EXOs and PD-1 on CD8⁺ cytotoxic T cells (F. L. Ricklefs et al., Sci. Adv. 4, eaar2766 (2018)). To determine whether SFX reduces exosomal PD-L1 through the regulation of EXO secretion, CT26 tumor-bearing mice received the following treatments (FIG. 3A): Dulbecco's phosphate buffered saline (DPBS), SFX, αPD-1, and SFX+αPD-1 (SFX dose: 200 mg/kg, αPD-1 dose: 5 mg/kg); exosomal PD-L1 in the mouse plasma in each group was evaluated using ELISA. As the basal exosomal PD-L1 level in wildtype mice are negligible compared to those in tumor-bearing mice, tumor-derived EXOs would primarily reflect the exosomal PD-L1 levels in tumor-bearing mice (FIG. 2). As shown in FIG. 4C, SFX alone significantly decreased an exosomal PD-L1 levels in the plasma, primarily due to inhibition of exosomal secretion. In contrast, αPD-1 alone markedly increased the exosomal PD-L1 level in the plasma, which may be due to the overexpression of PD-L1 in cancer cells stimulated by IFN-γ. This enhanced exosomal PD-L1 level, causing CD8⁺ cytotoxic T cell exhaustion, is responsible for the limited antitumor efficacy of αPD-1. Interestingly, when αPD-1 was combined with SFX, exosomal PD-L1 was restored to the basal level, comparable to that in the DPBS group. In addition, SFX+αPD-1 increased the secretion of IFN-γ, a representative inflammatory cytokine that is secreted by activated immune cells and plays an important role in antitumor immunity (FIG. 4D). Therefore, αPD-1 may elicit a strong antitumor immune response by minimizing exosomal PD-L1-mediated CD8⁺ cytotoxic T cell exhaustion in the presence of SFX.

### (2) SFX Synergistically Enhances the Antitumor Effect of Immune Checkpoint Inhibitors

To demonstrate the therapeutic efficacy of SFX in combination with αPD-1, CT26 colon cancer cells were used to generate a tumor-bearing mouse model. Murine CT26 cells were chosen for additional in vivo experiments because they overexpress ETA, and are available for cancer immunotherapy. To evaluate the effect of SFX on the antitumor response to αPD-1, CT26 colon cancer cells were used to generate a tumor-bearing mouse model (FIG. 3A): Dulbecco's phosphate buffered saline (DPBS), SFX, αPD-1, and SFX+αPD-1 (SFX dose: 200 mg/kg, αPD-1 dose: 5 mg/kg). As shown in FIG. 3B, tumor growth was effectively retarded by SFX, αPD-1, and SFX+αPD-1. Notably, compared to αPD-1, SFX+αPD-1 significantly reduced the tumor growth rate, indicating a synergistic anticancer effect by SFX-mediated inhibition of exosomal PD-L1. The data for tumor weights and images of the excised tumors were consistent with the results of the tumor volumes in the various groups (FIGS. 4, A and B).

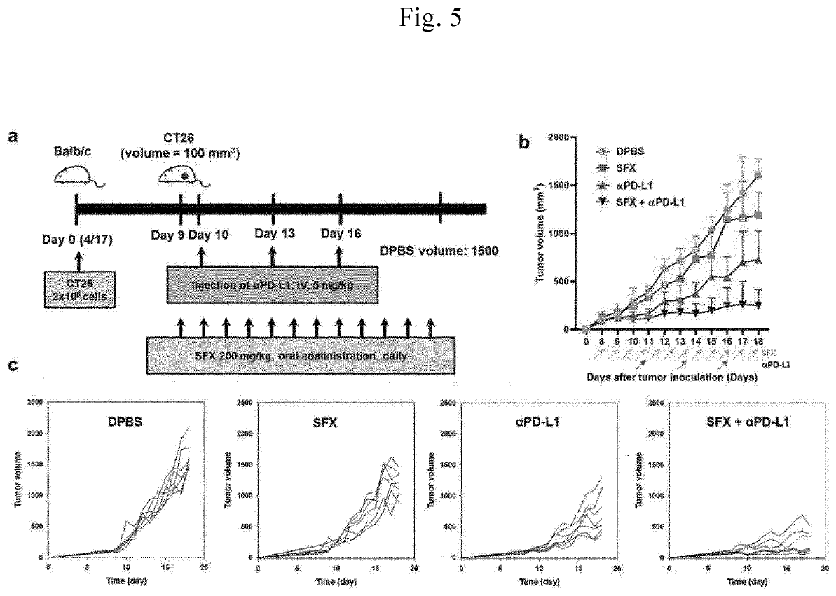

When considering the effect of SFX on the inhibition of exosomal secretion, αPD-L1 is another promising candidate for combination therapy. As shown in FIG. 5, the SFX+αPD-L1 group exhibited much higher antitumor efficacy than the SFX- or αPD-L1-treated groups. Therefore, αPD-L1 is expected to induce an enhanced antitumor immune response by avoiding exosomal PD-L1-mediated neutralization in the presence of SFX. Overall, SFX markedly enhanced the antitumor efficacy of immune checkpoint inhibitors by inhibiting exosomal secretion.

### (3) SFX Potentiates Antitumor Immune Response by Suppressing Exosomal PD-L1

Given that SFX reduced exosomal PD-L1 and elevated IFN-γ, we sought to evaluate the generation of antitumor immunity by analyzing the immune cells in the tumor microenvironment. To optimize the depletion effect of exosomal PD-L1, the therapeutic schedule was slightly modified by administering SFX on day 12, by which time sufficient immune cells could be obtained (FIG. 6A).

Tumor-infiltrating lymphocytes (TILs), which play a critical role in antitumor immune response, trigger the cancer immunity cycle by provoking apoptotic cell death and producing cancer-associated antigens. In this study, CD45⁺ TILs were isolated from the excised tumors to investigate the effect of SFX on the antitumor response to αPD-1. In the SFX+αPD-1 group, the number of CD4⁺ cells in the TME was comparable to that in the other groups, implying that CD4⁺ T cells were not a major subset mediating the synergistic effect of SFX and αPD-1 (FIGS. 6, B and C). Compared to DPBS, SFX+αPD-1 significantly increased CD8⁺ TILs in the TME (FIGS. 6, D and E). Further, αPD-1 increased the population of CD3⁺CD8⁺ cytotoxic T cells in the TME due to its specific binding to PD-1 on CD8⁺ cytotoxic T cells (FIGS. 6, F and G). Interestingly, compared to αPD-1 alone, SFX+αPD-1 significantly increased the levels of CD3⁺CD8⁺ TILs in the TME, implying that inhibition of exosomal PD-L1 enhances the bioactivity of αPD-1 to induce a strong anticancer immune response. Overall, SFX-mediated inhibition of tumor-derived exosomal PD-L1 enhanced the CD8⁺ T cell-mediated antitumor immune response.

### (4) ETA Antagonists Other than SFX Also Inhibit Cancer EXO Secretion

To evaluate the ability of ETA antagonists to inhibit exosome secretion, melanoma cell line B16F10 and colon cancer cell line CT26 (3×10⁶) were attached to a 150 pi dish, and after 24 hours, sulfisoxazole (SFX), ambrisentan (AMB), and macitentan (MCT) as ETA antagonists and GW2869 as a control were treated, respectively. After 24 hours, the supernatant was recovered, exosomes were extracted through continuous centrifugation, and then quantitatively evaluated using NTA.

As a result, as shown in FIG. 7, in the B16F10 cell line, which expresses the endothelin receptor (ETR) at a moderate level, all ETA antagonists inhibited exosome secretion at the level of 75% compared to the control group that was not treated with the drug. Further, in the CT26 cell line, which expresses ETR at a high level, it was confirmed that ETA antagonists inhibited exosome secretion by up to 40%.

In addition, colon cancer cell line CT26 (3×10⁶) was attached to a 150 pi dish, and after 24 hours, bosentan (BST), ambrisentan (AMB), and macitentan (MCT) as ETA antagonists were treated, respectively. After 24 hours, the supernatant was recovered, exosomes were extracted through continuous centrifugation, and then quantitatively evaluated using NTA.

As a result, as shown in FIG. 15, in the CT26 cell line, which expresses endothelin receptor (ETR) at high levels, all ETA antagonists inhibited exosome secretion by up to 40% compared to the control group that was not treated with the drug.

### (5) ETA Antagonists Other than SFX Also Synergistically Enhances the Antitumor Effect of Immune Checkpoint Inhibitors

To demonstrate the therapeutic efficacy of ETA antagonists other than SFX when combined with αPD-1, a tumor-bearing mouse model was generated using CT26 colon cancer cells. Murine CT26 cells were used for additional in vivo experiments because they overexpress ETA and can be used for cancer immunotherapy.

To evaluate the effect of bosentan (BST) on the antitumor response to αPD-1, CT26 tumor-bearing mice received the following treatments (FIG. 16A): Dulbecco's phosphate buffered saline (DPBS), BST, αPD-1, and BST+αPD-1. BST was administered orally daily at a dose of 10 mg/kg, and αPD-1 was diluted in physiological saline and injected intraperitoneally at a dose of 5 mg/kg every 3 days for a total of 3 times. The tumor volume was measured every day by measuring long-axis and short-axis of the tumor using calipers. As a result, as shown in FIG. 16B, tumor growth was effectively retarded by BST, αPD-1, and BST+αPD-1. Notably, compared to a control group, BST+αPD-1 caused the greatest reduction in cancer volume, and compared to αPD-1, BST+αPD-1 significantly reduced the tumor growth rate, indicating a synergistic anticancer effect by BST-mediated inhibition of exosomal PD-L1. The data for tumor weights of the excised tumors were consistent with the results of the tumor volumes in the various groups.

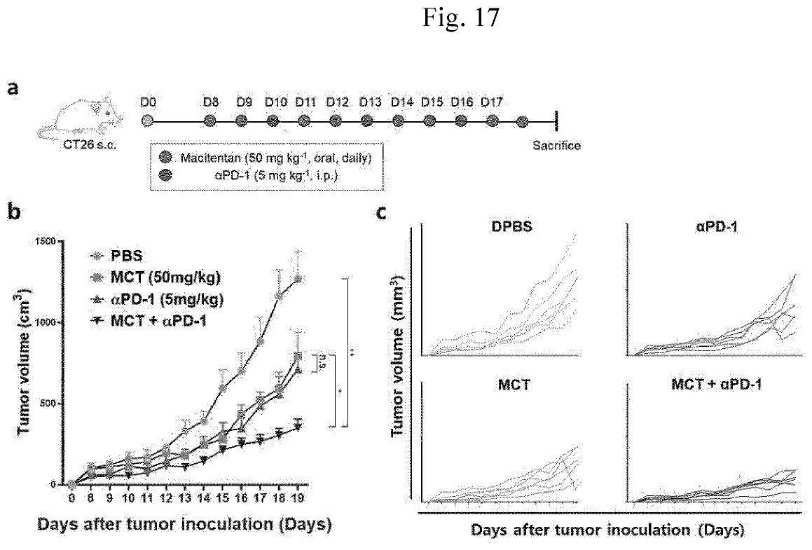

In addition, to evaluate the effect of macitentan (MCT) on the antitumor response to αPD-1, CT26 tumor-bearing mice received the following treatments (FIG. 17A): Dulbecco's phosphate buffered saline (DPBS), MCT, αPD-1, and MCT+αPD-1. MCT was administered orally daily at a dose of 50 mg/kg, and αPD-1 was diluted in physiological saline and injected intraperitoneally at a dose of 5 mg/kg every 3 days for a total of 3 times. The cancer volume was measured every day by measuring long-axis and short-axis of the tumor using calipers. As a result, as shown in FIG. 17B, tumor growth was effectively retarded by MCT, αPD-1, and MCT+αPD-1. Notably, compared to a control group, MCT+αPD-1 caused the greatest reduction in cancer volume, and compared to αPD-1, MCT+αPD-1 significantly reduced the tumor growth rate, indicating a synergistic anticancer effect by MCT-mediated inhibition of exosomal PD-L1. The data for tumor weights of the excised tumors were consistent with the results of the tumor volumes in the various groups.

### Example 2. Evaluation of Synergistic Effects by Administration of Conjugate of ETA Antagonist and Immune Checkpoint Inhibitor

### (1) The Preparation of a Conjugate Ab-VC-AMB

Fmoc-VC-AMB was prepared by stirring a peptide-based valine-citrulline (VC) linker (Fmoc-Val-Cit-PAB-OH, MedKoo) sensitive to an enzyme (Cathepsin B) overexpressed in a cancer microenvironment with an exosome secretion inhibitor ambrisentan (AMB) in the presence of 1-ethyl-3-(3-dimethylaminopropyl) carbodiimide-hydrochloride (EDC-HCl) and a 4-dimethylaminopyridine (DMAP) catalyst to form an ester bond. VC-AMB was prepared by removing Fmoc from the prepared Fmoc-VC-AMB in the presence of piperidine. Mal-VC-AMB was prepared by chemically conjugating a spacer for antibody conjugation (6-maleimidohexanoic acid, Tokyo Chemical Industry) to the prepared VC-AMB. After an antibody was reduced by treating tris(2-carboxyethyl)phosphine (TCEP) with a pH 8.0 borate buffer at 25° C. for 30 minutes, VC-AMB was chemically conjugated to a PD-1 antibody (BioXCell) by adding a cold 20% acetonitrile solution to 1.1 eq Mal-VC-AMB of a free thiol group of an antibody determined through 5,5′dithiobis (2-nitrobenzoic acid (DTNB) at 4° C. Thereafter, the reaction was stopped by adding an excessive amount of cysteine thereto, and then an antibody-drug conjugate was obtained using a Zeba desalting column (Thermo) and was named Ab-VC-AMB or ADC (antibody-drug conjugate).

### (2) Evaluation of Therapeutic Efficacy of Ab-VC-AMB in Disease Animal Model

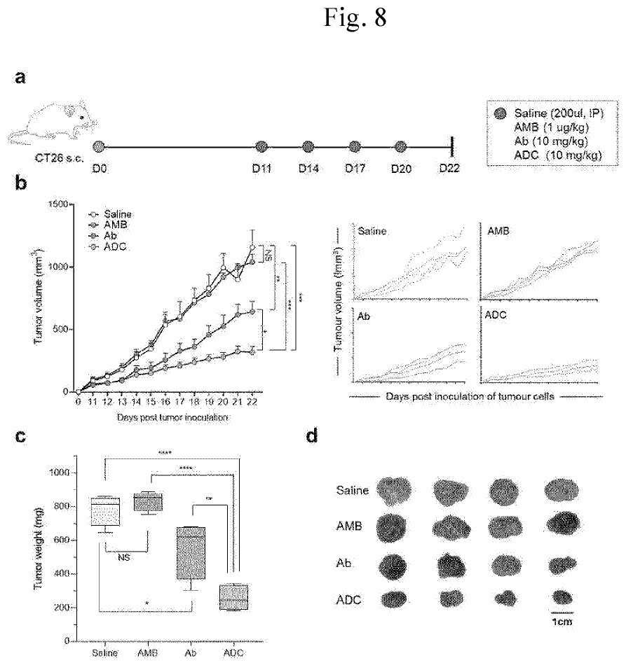

In order to evaluate the therapeutic efficacy of Ab-VC-AMB in a disease animal model, a cancer animal model was produced by subcutaneously inoculating CT26 (1×10⁶ cells), which is a colon cancer cell line, into mice and allowing tumors to grow for 10 days (FIG. 8A). Thereafter, on days 11, 14, 17 and 20, Ab-VC-AMB, saline, PD-L1 antibody (Ab) or AMB was injected intraperitoneally into the cancer animal model (Ab 10 mg/kg, AMB 1 μg/kg, Ab-VC-AMB 10 mg/kg), and then the therapeutic efficacy was evaluated for 11 days.

As a result, as shown in FIG. 8B-D, in the case of an Ab-VC-AMB experimental group, the cancer volume was at the level of 27% compared to the control to which saline was administered, cancer growth was remarkably suppressed, and the volume of cancer was about 49% even compared to the Ab control, showing high cancer therapeutic efficacy. In addition, compared to the group administered Ab or AMB alone, Ab-VC-AMB experimental group showed a clear synergistic treatment effect.

### (3) Evaluation of Ability of Ab-VC-AMB to Inhibit Exosome Secretion in Disease Animal Model

To evaluate the ability of Ab-VC-AMB to inhibit the secretion of exosomes, after the animal model for which the therapeutic efficacy evaluation was completed was sacrificed, plasma was isolated, and exosomes were isolated and extracted using an exosome isolation kit (Invitrogen total exosome isolation reagent). BCA analysis (bicinchoninic acid assay) was performed on the isolated exosomes, and PD-L1 on the surface of the exosomes was quantified through ELISA analysis. A 96-well plate was coated with 2 g/ml PD-L1 antibody by incubating at 4° C. for 16 hours at room temperature. After washing the plate three times with phosphate-buffered saline with 0.05% Tween 20 (PBST), blocking buffer was added thereto and the plate was incubated at room temperature for 2 hours. After the plate was washed 3 times with PBST, standards and samples using the serially diluted PD-L1 antibody were placed and left to stand at room temperature for 2 hours. A biotinylated PD-L1 detection antibody was added thereto, and the plate was incubated at room temperature for 2 hours. The plate was washed 3 times again, 40-fold-diluted streptavidin-conjugated peroxidase (Streptavidin-HRP) was added thereto, and the plate was incubated at room temperature for 20 minutes. After the plate was washed 3 times with PBST, a substrate solution in which H₂O₂ and tetramethylbenzidine were mixed at 1:1 was added to each well, and after the plate was incubated for 20 minutes, and the reaction was stopped by adding 2N H₂SO₄ thereto. Absorbance at 450 nm was measured using a microplate reader.

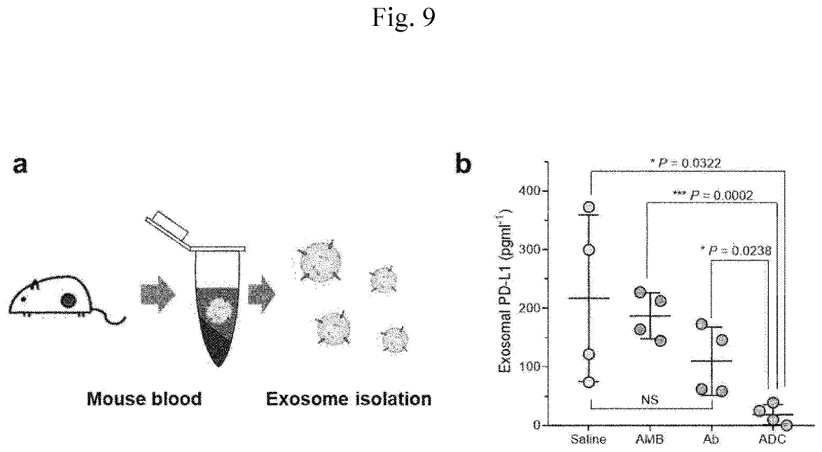

As a result, as shown in FIG. 9, the exosomal PD-L1 (PD-L1) level of exosomes showed a tendency to decrease remarkably in the Ab-VC-AMB experimental group compared to the other groups. Such results mean that after the AMB drug was released from Ab-VC-AMB in response to the cancer microenvironment, the secretion of cancer exosomes was effectively inhibited, and are results supporting the high therapeutic efficacy of the Ab-VC-AMB experimental group in an anti-cancer treatment efficacy experiment through the disease animal model.

### Example 3. Preparation of a Conjugate of ETA Antagonist and Biocompatible Polymer and Evaluation of Ability to Inhibit Exosome Secretion

### (1) Preparation of a Conjugate of ETA Antagonist and Biocompatible Polymer-1

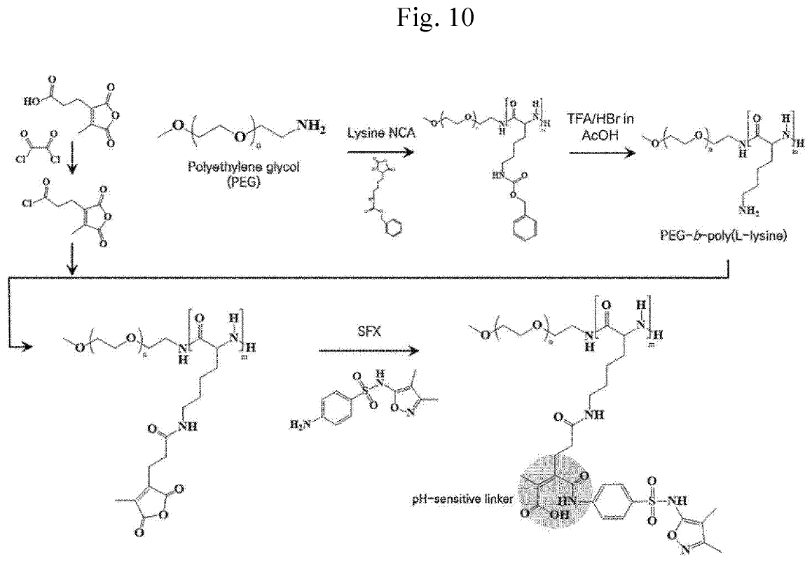

A conjugate (polymer-drug conjugate (PDC)) of the ETA antagonist sulfisoxazole and a biocompatible polymer was synthesized as shown in FIG. 10.

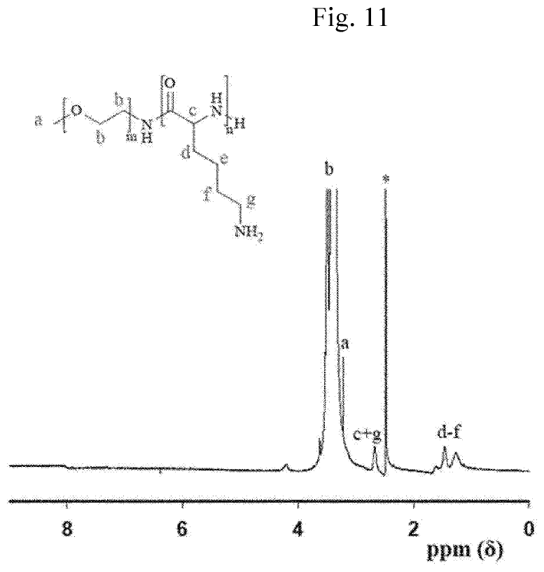

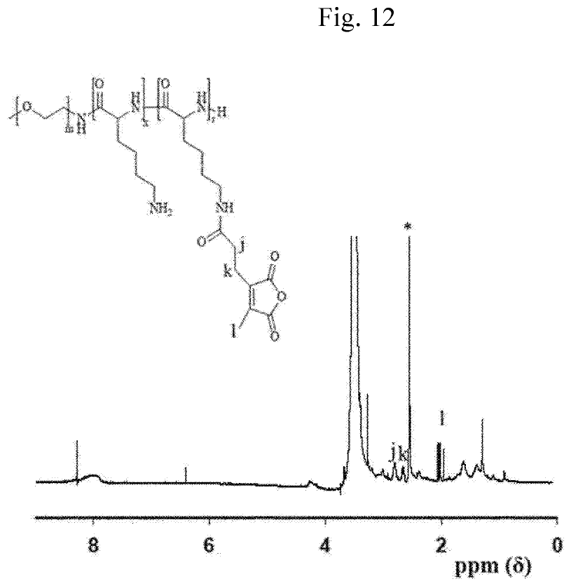

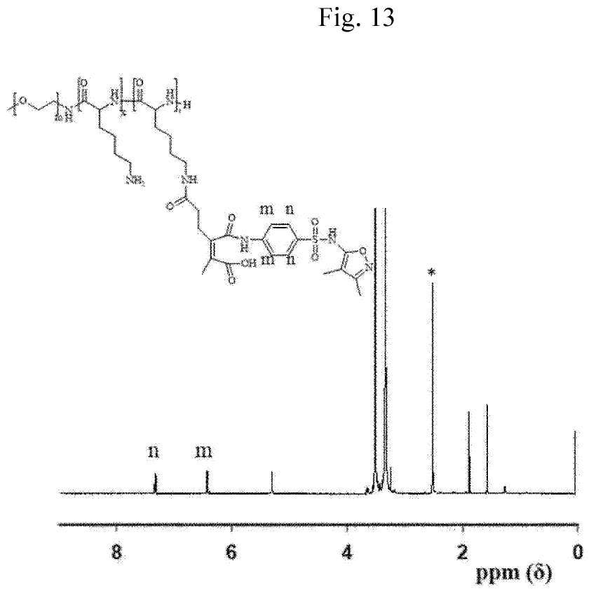

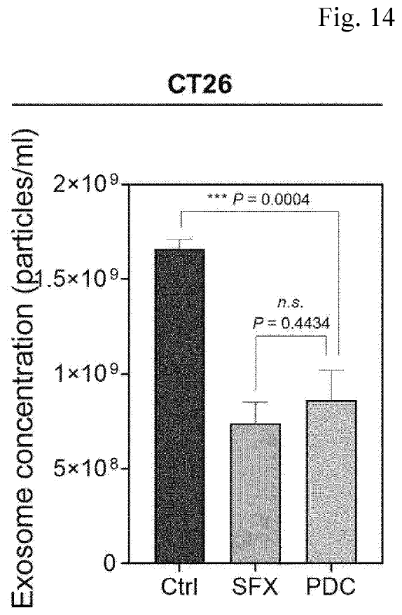

Specifically, using tetrahydrofuran as a solvent, N₆—Carbobenzyloxy-L-lysine (Sigma) and triphosgene (Tokyo Chemical Industry) were reacted at 50° C. for 3 hours to prepare N₆-Carbobenzyloxy-L-lysine N-carboxyanhydride (Lysine NCA). Using dimethylformamide as a solvent, methoxypolyethylene glycol amine (PEG amine, 5 kDa, LaysanBio) and 20 eq of Lysine NCA were reacted at 35° C. for 24 hours. To remove the carbobenzyloxy group of the reactant, hydrogen bromide solution and the sample were reacted at room temperature for 2 hours using trifluoroacetic acid as a solvent, thereby producing PEG-b-Poly(L-lysine) block copolymer, which was confirmed by ¹H NMR (FIG. 11). To introduce a pH-responsive linker that can be hydrolyzed in a mildly acidic environment to release the drug, 3-(4-Methyl-2,5-dioxo-2,5-dihydrofuran-3-yl)propanoic acid (CDM, Ambeed) and oxalyl chloride were reacted at room temperature using dicholoromethane as a solvent and then dried in vacuum to prepare acyl chloride CDM (CDM-Cl). Afterwards, CDM-Cl and PEG-b-Poly(L-lysine) were reacted at room temperature for 24 hours under the presence of a pyridine catalyst to prepare the linker-introduced polymer PEG-b-Poly (L-lysine-CDM), which was confirmed by ¹H NMR (FIG. 12). PEG-b-Poly(L-lysine-CDM-SFX) was prepared through a triethylamine-catalyzed reaction between this sample and sulfisoxazole (SFX, Tokyo Chemical Industry) and confirmed by ¹H NMR (FIG. 13).

As SFX has an intrinsic absorbance at 270 nm, the SFX content in PDC was measured using a UV-Vis spectrophotometer. As a result, the SFX content of the produced PDC was 4 wt %.

### (2) Preparation of a Conjugate of ETA Antagonist and Biocompatible Polymer-2

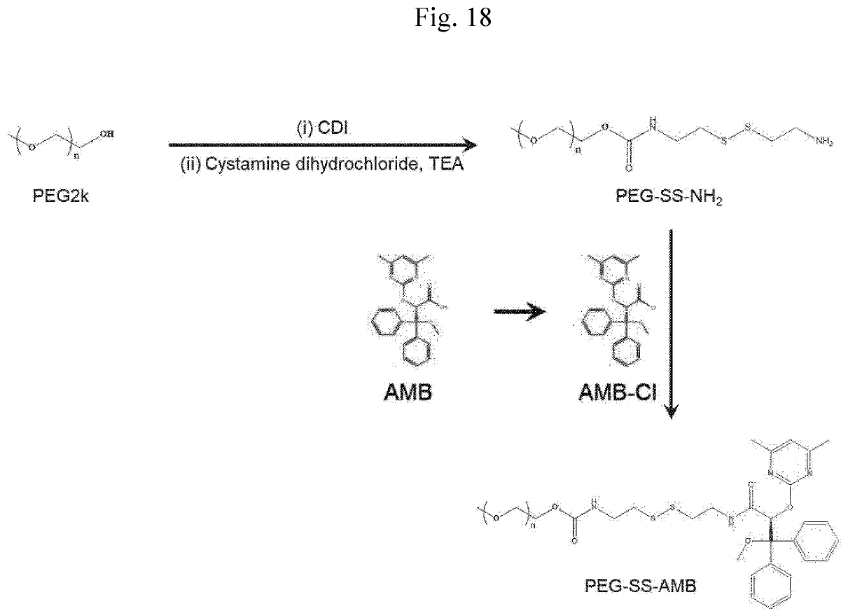

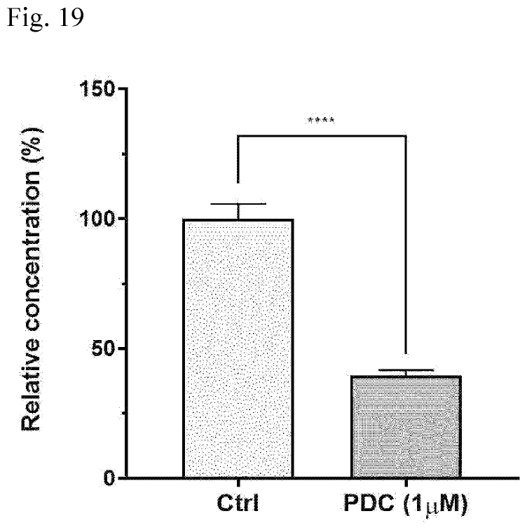

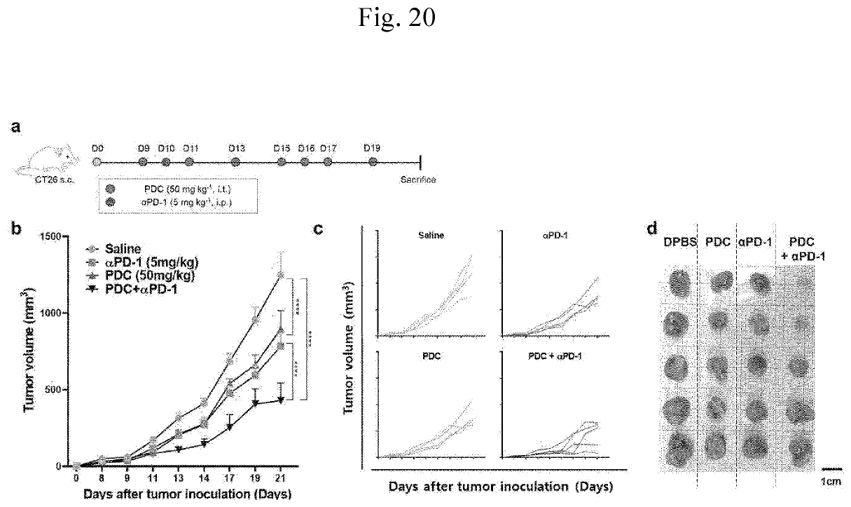

A conjugate (PDC) of the ETA antagonist ambrisentan and a biocompatible polymer was synthesized as shown in FIG. 18.

Specifically, using dimethylforamide as a solvent, polyethylene glycol 1500 monomethyl ether (mPEG-OH, 1.5 kDa, Sigma) and 10 eq of carbonyldiimidazole (CDI, Sigma) were reacted at room temperature for 24 hours. The reactant was added to an excess of diethyl ether, and the resulting precipitate was filtered through filter paper and dried under vacuum to prepare PEG-CDI. Afterwards, PEG-CDI and 10 eq of cystamine dihydrochloride (Sigma) were reacted at room temperature for 24 hours under a triethylamine catalyst, and then purified by dialysis to prepare PEG-SS-NH₂ into which a disulfide linker was introduced.

Using dichloromethane as a solvent, AMB and oxalyl chloride were reacted at room temperature and then dried in vacuum to prepare acyl chloride AMB (AMB-Cl). Afterwards, AMB-Cl and PEG-SS-NH₂ were reacted at room temperature for 24 hours under a pyridine catalyst to prepare PEG-SS-AMB.

### (3) Evaluation of the Ability of PDC to Inhibit Exosome Secretion

To evaluate the ability of PEG-b-Poly(L-lysine-CDM-SFX) to inhibit the secretion of exosomes, CT26 cells, a murine colon cancer cell line, were treated with 100 M of SFX and the same amount of PDC, exosomes from the supernatant were separated, and secreted exosomes were quantitatively analyzed. As a result, SFX inhibited exosome secretion by more than 50% compared to the untreated control group, and PEG-b-Poly (L-lysine-CDM-SFX) also showed an exosome inhibition effect equivalent to SFX (FIG. 14).

In addition, to evaluate the ability of PEG-SS-AMB to inhibit the secretion of exosomes, CT26 cells, a murine colon cancer cell line, were treated with 1 M of PEG-SS-AMB, exosomes from the supernatant were separated, and secreted exosomes were quantitatively analyzed. As a result, as shown in FIG. 19, PEG-SS-AMB inhibited exosome secretion by more than 50% compared to the control group that was not treated with the drug.

### (4) Evaluation of Therapeutic Efficacy of PDC in Disease Animal Model

In order to evaluate the therapeutic efficacy of PEG-SS-AMB in a disease animal model, a cancer animal model was produced by subcutaneously inoculating CT26 (1×10⁶ cells), which is a colon cancer cell line, into mice. Afterwards, therapeutic efficacy was evaluated by injecting saline, PEG-SS-AMB, αPD-1 antibody, or PEG-SS-AMB+αPD-1 through direct intratumoral administration (αPD-1 5 mg/kg, PEG-SS-AMB 50 mg/kg).

As a result, as shown in FIG. 20, in the case of the PEG-SS-AMB experimental group, the cancer volume was suppressed compared to the control group to which saline was administered. In the case of the PEG-SS-AMB+αPD-1 combination group, the cancer volume was at the level of 35% compared to the control to which saline was administered, cancer growth was remarkably suppressed, and the volume of cancer was about 55% even compared to the αPD-1 administration group, showing high cancer therapeutic efficacy. Through this, it was confirmed that compared to the group administered αPD-1 or PEG-SS-AMB alone, the PEG-SS-AMB+αPD-1 combination group showed a clear synergistic treatment effect.

Based on the above description, it will be understood by those skilled in the art that the present disclosure may be implemented in a different specific form without changing the technical spirit or essential characteristics thereof. In this regard, it should be understood that the above embodiment is not limitative, but illustrative in all aspects. The scope of the disclosure is defined by the appended claims rather than by the description preceding them, and therefore all changes and modifications that fall within metes and bounds of the claims, or equivalents of such metes and bounds, are intended to be embraced by the claims.

1. A method of preventing or treating cancer, comprising administering to a subject in need thereof a therapeutically effective amount of an endothelin receptor type A (ETA) antagonist and a therapeutically effective amount of an immune checkpoint inhibitor, wherein the ETA antagonist and the immune checkpoint inhibitor are administered simultaneously, separately, or sequentially.

2. The method of claim 1, wherein the ETA antagonist is selected from the group consisting of ambrisentan, sulfisoxazole, macitentan, BQ-123, BQ-788, zibotentan, sitaxentan, atrasentan, bosentan, tezosentan and A192621.

3. The method of claim 1, wherein the immune checkpoint inhibitor is an antibody that specifically binds to PD-1 or PD-L1, or an antigen-binding fragment thereof.

4. The method of claim 1, wherein the ETA antagonist is in the form of a conjugate conjugated to a biocompatible polymer.

5. The method of claim 4, wherein the biocompatible polymer is a polymer comprising a nonionic hydrophilic polymer moiety, a polymer comprising an ionic polymer moiety, or a copolymer comprising both.

6. The method of claim 5, wherein the nonionic hydrophilic polymer is polyethylene glycol, polypropylene glycol, polyoxazoline, polyvinylpyrrolidone, polyvinyl alcohol, polyacrylamide, polymethacrylamide, polyacrylic acid ester, polymethacrylic acid ester, polyhydroxyethyl methacrylate, dextran, polysaccharide, or methylcellulose.

7. The method of claim 5, wherein the ionic polymer is poly(L-lysine), polyaspartic acid, poly(L-glutamic acid), polyornithine, polyarginine, polyhomoarginine, polyhistidine, hyaluronic acid, alginic acid, polyacrylic acid, polymethacrylic acid, chitosan, polyethyleneimine, polyvinyl phosphate, polyethylene glycol methacrylate phosphate, carboxymethylcellulose, or heparin.

8. The method of claim 5, wherein the ETA antagonist is conjugated to the biocompatible polymer via a linker or is conjugated to the biocompatible polymer via a pH-sensitive linker or an acid-labile linker.

9. The method of claim 5, wherein the copolymer is a block copolymer or a graft copolymer.

10. The method of claim 1, wherein the ETA antagonist is in the form of a conjugate conjugated to the immune checkpoint inhibitor.

11. The method of claim 10, wherein the ETA antagonist is conjugated to the immune checkpoint inhibitor via a linker or is conjugated to the immune checkpoint inhibitor via a cleavable linker that is cleaved by a protease.

12. A method of preventing or treating cancer, comprising administering to a subject in need thereof a therapeutically effective amount of a conjugate of an endothelin receptor type A (ETA) antagonist conjugated to a biocompatible polymer and a therapeutically effective amount of an immune checkpoint inhibitor, wherein the conjugate and the immune checkpoint inhibitor are administered simultaneously, separately, or sequentially.

13. The method of claim 12, wherein the biocompatible polymer is a polymer comprising a nonionic hydrophilic polymer moiety, a polymer comprising an ionic polymer moiety, or a copolymer comprising both.

14. The method of claim 13, wherein the nonionic hydrophilic polymer is polyethylene glycol, polypropylene glycol, polyoxazoline, polyvinylpyrrolidone, polyvinyl alcohol, polyacrylamide, polymethacrylamide, polyacrylic acid ester, polymethacrylic acid ester, polyhydroxyethyl methacrylate, dextran, polysaccharide, or methylcellulose.

15. The method of claim 13, wherein the ionic polymer is poly(L-lysine), polyaspartic acid, poly(L-glutamic acid), polyornithine, polyarginine, polyhomoarginine, polyhistidine, hyaluronic acid, alginic acid, polyacrylic acid, polymethacrylic acid, chitosan, polyethyleneimine, polyvinyl phosphate, polyethylene glycol methacrylate phosphate, carboxymethylcellulose, or heparin.

16. The method of claim 13, wherein the copolymer is a block copolymer or a graft copolymer.

17. The method of claim 13, wherein the ETA antagonist is conjugated to the biocompatible polymer via a linker or is conjugated to the biocompatible polymer via a pH-sensitive linker or an acid-labile linker.

18. A method of preventing or treating cancer, comprising administering to a subject in need thereof a therapeutically effective amount of a conjugate of an endothelin receptor type A (ETA) antagonist conjugated to an immune checkpoint inhibitor.

19. The method of claim 12, wherein the ETA antagonist is selected from the group consisting of ambrisentan, sulfisoxazole, macitentan, BQ-123, BQ-788, zibotentan, sitaxentan, atrasentan, bosentan, tezosentan and A192621.

20. The method of claim 12, wherein the immune checkpoint inhibitor is an antibody that specifically binds to PD-1 or PD-L1, or an antigen-binding fragment thereof.

21. The method of claim 18, wherein the ETA antagonist is selected from the group consisting of ambrisentan, sulfisoxazole, macitentan, BQ-123, BQ-788, zibotentan, sitaxentan, atrasentan, bosentan, tezosentan and A192621.

22. The method of claim 18, wherein the immune checkpoint inhibitor is an antibody that specifically binds to PD-1 or PD-L1, or an antigen-binding fragment thereof.
